(19) United States
(12) Patent Application Publication
(10) Pub. No.: US 2025/0284114 A1
(43) Pub. Date: Sep. 11, 2025

(54) IMAGE SENSING DEVICE FOR SIDE-VIEWING ENDOSCOPE

(71) Applicant: Medimaging Integrated Solution, Inc., Hsinchu (TW)

(72) Inventors: SHANGYI WU, Hsinchu (TW); HSIN-HSIEN HSIEH, Hsinchu (TW)

(21) Appl. No.: 18/939,809

(22) Filed: Nov. 7, 2024

(30) Foreign Application Priority Data
Mar. 7, 2024 (TW) 113202333

(51) Int. Cl. G02B 23/24; A61B 1/00; H04N 23/50; H04N 23/56

(52) U.S. Cl. CPC G02B 23/2484; G02B 23/2461; H04N 23/555; H04N 23/56; A61B 1/00177

(57) ABSTRACT

An image sensing device for a side-view endoscope includes a substrate having a first surface, wherein a wire connection member, an image module connection member and a light source module connection member are disposed on the first surface, and wherein the wire connection member is electrically connected with the image module connection member and the light source module connection member; an image module electrically connected with the image module connection member and configured to capture external images along a lateral-side direction of a distal tip; and a light source module electrically connected with the light source module connection member, configured to project a light source along the lateral-side direction of the distal tip. By disposing the image module and the light source module on the same surface of the substrate, the device can be applied to side-view endoscopes and has the competitive advantage of miniaturization.

### BACKGROUND OF THE INVENTION

### 1. Field of the Invention

The present invention relates to an endoscope, particularly to an image sensing device for a side-viewing endoscope.

### 2. Description of the Prior Art

Endoscopes have been widely used in industry and medicine. In industry, endoscopes may be used to inspect a narrow space, such as interiors of channels or complicated mechanical structures, without dismantling these structures, whereby to assist in inspection, improve product yield, and prolong the service lives of machines.

In medicine, endoscopes are often used in minimum invasive surgery and health examination, whereby to decrease invasiveness and avoid tissue damage. The smaller the endoscope, the better the feeling of the patient, and the fast the wound repair.

However, the size of endoscopes is hard to reduce at present, especially the front-viewing endoscopes.

### SUMMARY OF THE INVENTION

One objective of the present invention is to provide an image sensing device for a small-size side-viewing endoscope.

The preset invention proposes an image sensing device for a side-viewing endoscope, which is disposed in a distal tip of the endoscope and comprises a substrate, a connection wire, an image module, and at least one light source module. The substrate includes a first surface and a second surface opposite to the first surface. The substrate further includes a wire connection member, an image module connection member and at least one light source module connection member, which are all arranged on the first surface. The wire connection member is electrically connected with the image module connection member. The wire connection member is also electrically connected with the light source module connection member. The image module captures external images along a lateral-side direction, which is between two ends of the distal tip. The light source module is electrically connected with the light source module connection member, projecting light along the lateral-side direction of the distal tip.

It is learned from the above description: the present invention improves the endoscope from the conventional front-viewing type into a side-viewing type. In other words, the image module captures images along the lateral-side direction of the distal tip. Further, the present invention provides a design able to minimize the overall size: disposing the connection wire, the image module and the light source module all on the first surface of the substrate. Thereby, the size of an endoscope is effectively reduced, and the objective of the present invention is achieved.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein

FIG. 1 is a perspective view schematically an appearance of an image sensing device for a side-viewing endoscope according to one embodiment of the present invention;

FIG. 2 is a perspective view schematically an image sensing device for a side-viewing endoscope according to one embodiment of the present invention;

FIG. 3 is a perspective view schematically a substrate of an image sensing device for a side-viewing endoscope according to one embodiment of the present invention;

FIG. 4 is a perspective view schematically substrates, image modules and light source modules of an image sensing device for a side-viewing endoscope according to one embodiment of the present invention;

FIG. 5 is a perspective view schematically a light source module of an image sensing device for a side-viewing endoscope according to one embodiment of the present invention;

FIG. 6 is a perspective view schematically a light source module of an image sensing device for a side-viewing endoscope according to another embodiment of the present invention;

FIG. 7 is a perspective view schematically a light shield of an image sensing device for a side-viewing endoscope according to another embodiment of the present invention;

FIG. 8 is a perspective view schematically substrates, image modules, light source modules and light shields of an image sensing device for a side-viewing endoscope according to one embodiment of the present invention;

FIG. 9 is a perspective view schematically a substrate, an image module, light source modules, a light shield, and a sensor of an image sensing device for a side-viewing endoscope according to one embodiment of the present invention;

FIG. 10 is a perspective view schematically a substrate, an image module, light source modules, a light shield, a sensor and an optical protection layer of an image sensing device for a side-viewing endoscope according to one embodiment of the present invention;

FIG. 11 is a perspective view schematically an image sensing device for a side-viewing endoscope according to another embodiment of the present invention; and

FIG. 12 is a perspective view schematically an image sensing device for a side-viewing endoscope according to yet another embodiment of the present invention.

### DESCRIPTION OF THE PREFERRED EMBODIMENTS

The embodiments of the present invention will be further demonstrated in details hereinafter in cooperation with the corresponding drawings. In the drawings and the specification, the same numerals represent the same or the like elements as much as possible. For simplicity and convenient labelling, the shapes and thicknesses of the elements may be exaggerated in the drawings. It is easily understood: the elements belonging to the conventional technologies and well known by the persons skilled in the art may be not particularly depicted in the drawings or described in the specification. Various modifications and variations made by the persons skilled in the art according to the contents of the present invention are to be included by the scope of the present invention.

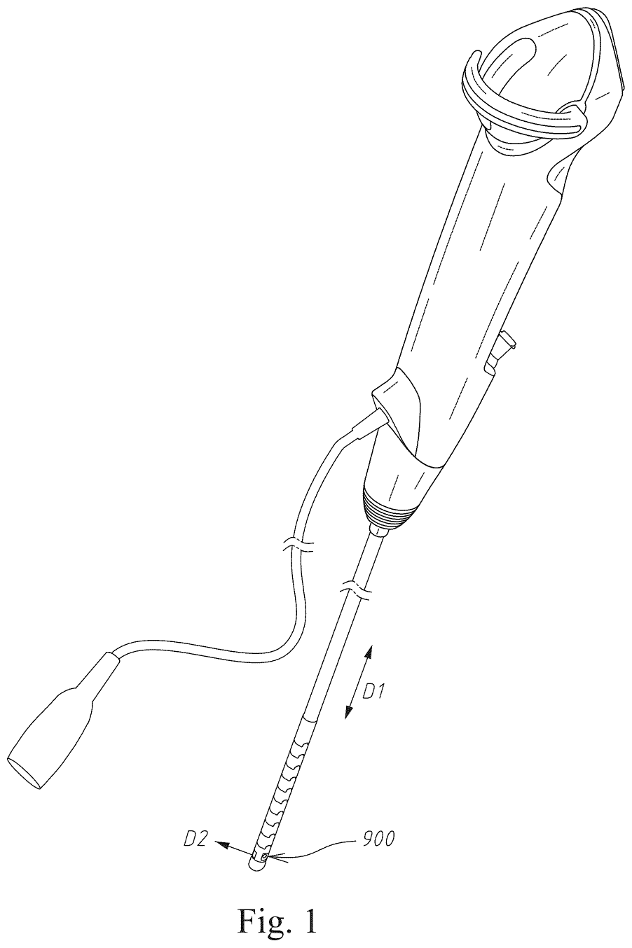

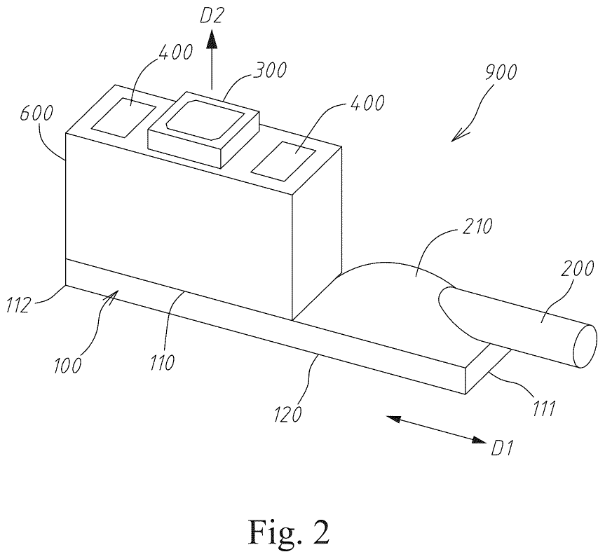

Refer to FIG. 1 and FIG. 2. In some embodiments of the present invention, the image sensing device for a side-viewing endoscope is disposed in a distal tip 900 of the endoscope and comprises a substrate 100, a connection wire 200, an image module 300, and at least one light source module 400.

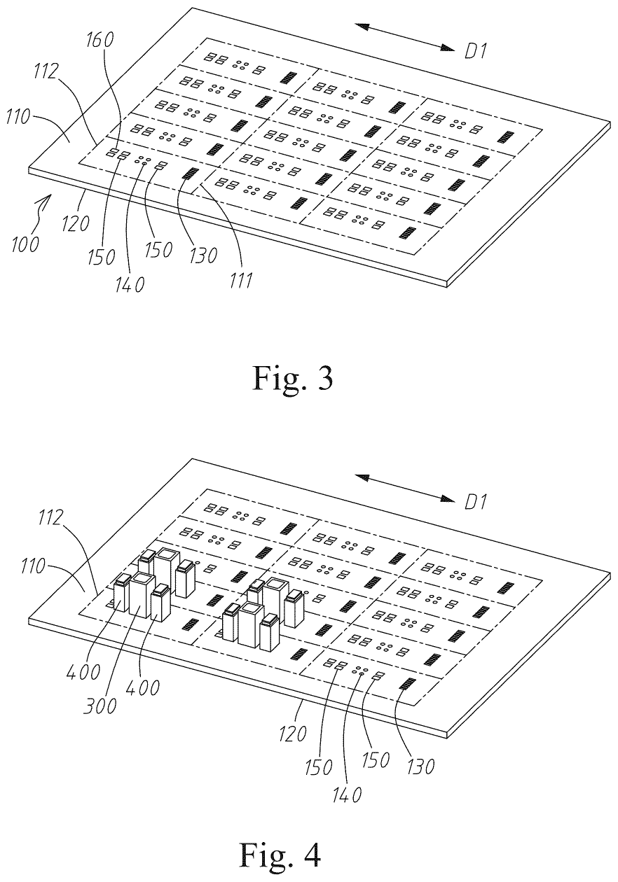

Refer to FIG. 3. The substrate 100 includes a first surface 110 and a second surface 120 opposite to the first surface 110. In the embodiments, the substrate 100 is a ceramic substrate, a printed circuit board (PCB), or a flexible printed circuit (FPC). In the embodiments, the substrate 100 functions as a carrier. The substrate 100 further includes a wire connection member 130, an image module connection member 140, and at least one light source module connection member 150, which are all arranged on the first surface 110. The wire connection member 130 is electrically connected with the image module connection member 140. The wire connection member 130 is also electrically connected with the light source module connection member 150. In fabricating the image sensing devices, the substrates 100, which have not be separated, are arranged in array, whereby to simplify the fabrication process and favor mass production. In addition to a rectangular shape, the substrate 100 may alternatively have another shape to meet different requirements.

In some embodiments, the wire connection member 130, the image module connection member 140 and the at least one light source module connection member 150 are arranged along a first direction D1. The first direction D1 is a direction along a first end 111 of the first surface 110 and a second end 112 of the first surface 110. Thus, the wire connection member 130, the image module connection member 140 and the at least one light source module connection member 150 are compactly arranged along the first direction D1. Thereby, the image sensing device and the distal tip 900, which are at the front end of the endoscope, may be diametrically minimized in a lateral-side directional D2 of the distal tip 900. It should be noted: the lateral-side directional D2 of the distal tip 900 is a direction parallel to the lateral side of the distal tip 900 and substantially vertical to the first direction D1, as shown in FIG. 2.

In some embodiments, the present invention may use a single light source module connection member 150. Along the first direction D1, the light source module connection member 150 is disposed on the first surface 110 and at one side of the image module connection member 140, which is corresponding to the first end 111; the wire connection member 130 is disposed on the first surface 110 and at one side of the image module connection member 140, which is corresponding to the second end 112. In other embodiments, along the first direction D1, the light source module connection member 150 is disposed on the first surface 110 and at one side of the image module connection member 140, which is corresponding to the second end 112; the wire connection member 130 is disposed on the first surface 110 and at one side of the image module connection member 140, which is corresponding to the first end 111.

In some embodiments, the present invention may use two light source module connection members 150. Along the first direction D1, the light source module connection members 150 are disposed on the first surface 110 and respectively at one side of the image module connection member 140, which is corresponding to the first end 111 and at one side of the image module connection member 140, which is corresponding to the second end 112. The wire connection member 130 is disposed on the first surface 110 between the light source module connection member 150 and the first end 111. Alternatively, the wire connection member 130 is disposed on the first surface 110 between the light source module connection member 150 and the second end 112.

Refer to FIG. 1 and FIG. 4. The image module 300 normally includes an optical lens module. The image module 300 captures external images along the lateral-side direction D2 of the distal tip 900. The image module 300 is joined and fixed to the image module connection member 140 in a high-temperature joining method. The image module 300 is electrically connected with and installed on the image module connection member 140 through the electrodes on the bottom thereof (BGA (Ball Grid Array) herein).

In some embodiments, the light source module connection members 150 are disposed on the first surface 110 and at either side or both sides of the image module connection member 140. The substrate 100 may have one, two, three or more light source module connection members 150. The light source module 400 may project light along the lateral-side direction D2 of the distal tip 900.

Refer to FIG. 3 and FIG. 4. In some embodiments, the image sensing device includes two light source module connection members 150. Two pairs of the light source module connection members 150 and the light source modules 400 are respectively disposed on left and right sides of the image module 300. The light source module 400 and the corresponding light source module connection member 150 are joined and secured in a high-temperature joining method, whereby the light source module 400 is electrically connected with and installed on the light source module connection member 150.

The light source module 400 may include a fluorescent powder layer 410 and a light-emitting element for emitting white light. Alternatively, the light source module 400 may be a light-emitting diode. In some embodiments, the light source module 400 may have light-emitting elements able to emit respectively lights with different wavelength; the wire connection member 130 has control wires connected with the light-emitting elements; the control wires receive control signals to turn on/off the light-emitting elements respectively emitting lights of different wavelengths.

The light source module 400 may emit light of the same wavelength and the same color temperature or emit lights of different wavelengths and different color temperatures, such as white light or infrared light. The lights of different wavelengths and different color temperatures may be turned on simultaneously or separately to meet different applications of the image sensing device.

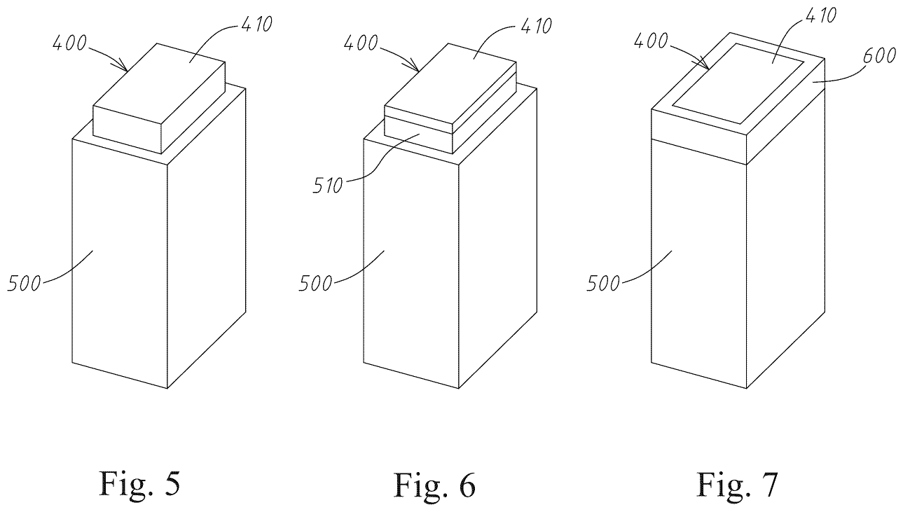

Refer to FIG. 5. In some embodiments, the image sensing device further includes a carrier 500, which is disposed between the light source module 400 and the light source module connection member 150. The carrier 500 works to let the light source module 400 have different heights. The relative height of the light source module 400 and the image module 300 may be accurately controlled through using the carriers 500 having different heights. The size of the carrier 500 may be obtained via simulation and test.

Refer to FIG. 6. In some embodiments, the image sensing device further includes an intermediate carrier 510, which is disposed between the carrier 500 and the light source module 400.

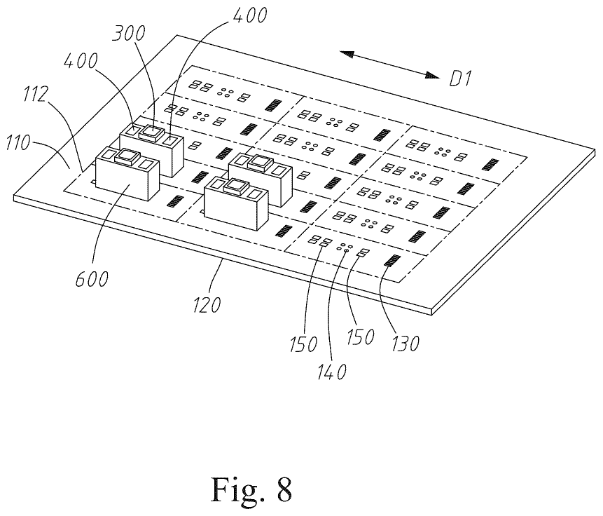

Refer to FIG. 7. In some embodiments, the image sensing device further includes a light shield 600, which is disposed between the light source module 400 and the image module 300. In some embodiments, the light shield 600 is disposed around the light source module 400. In some embodiments, the light shield 600 is disposed around the image module 300. Refer to FIG. 8. In some embodiments, the light shield 600 surrounds the light source module 400 and the image module 300.

It should be mentioned particularly: the light shield 600 may be made of a light-absorptive material, a light-reflective material, a light-scattering material, or a light-diffusive material. The abovementioned materials can prevent stray light from entering the image module 300 as well as homogenize the light source. The light shield 600 neither shades the top regions of the light source module 400 and the image module 300 nor shades the surrounding connection members. For example, the light shield 600 does not shade the wire connection member 130 lest the light generation efficiency, the image capture efficiency and the electric connection efficiency be affected.

The material of the light shield 600 may be filled into the space between the image module 300 and the light source module 400 and the surrounding regions to prevent from stray light and protect the image module 300 and the light source module 400.

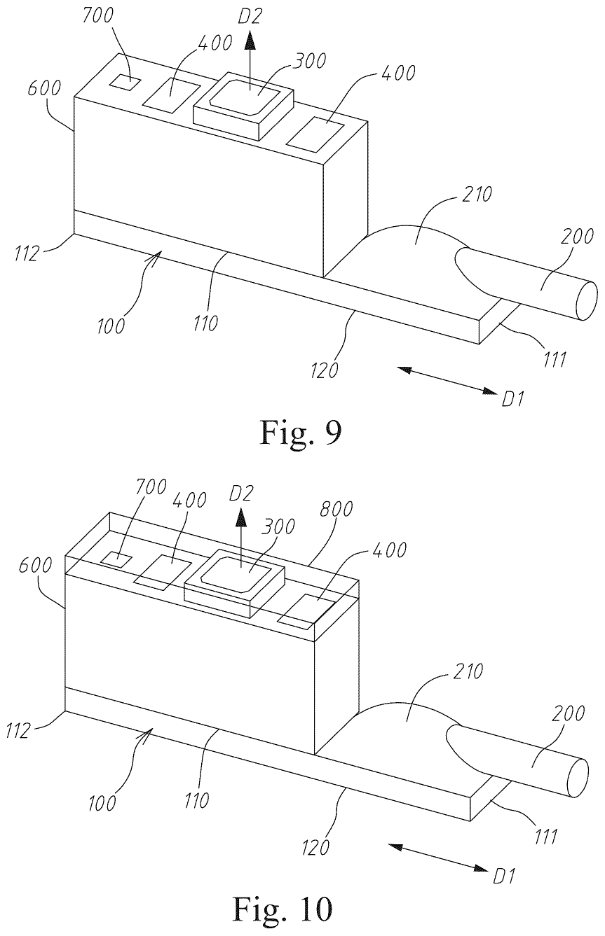

Refer to FIG. 9. In some embodiments, the image sensing device further includes a sensor 700, such as a pressure sensor or a thermosensitive sensor. The sensor 700 is electrically connected with a sensor connection member 160 on the first surface 110 of the substrate 100, as shown in FIG. 3. The sensor connection member 160 is electrically connected with the wire connection member 130. The sensor 700 is used to detect the temperature or pressure of the image sensing device so as to provide more information for the operator.

Refer to FIG. 10. In some embodiments, an optical protection layer 800 is formed on the surface of the light source module 400. The optical protection layer 800 covers the light source modules 400, but it does not cover the image module 300.

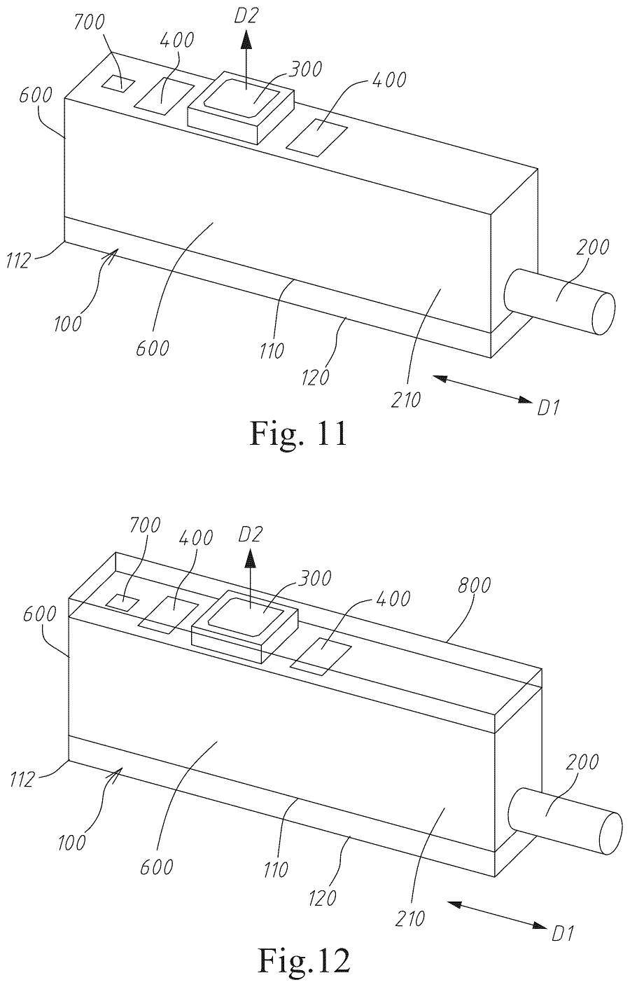

Refer to FIG. 11. In some embodiments, the connection wire 200 is electrically connected with the wire connection member 130 in a high-temperature soldering method. The connection wire 200 may be disposed and extended out according to requirement, whereby the size of the image sensing device may be minimized. Because the connection wire 200 is directly soldered to the wire connection member 130 without using an adapter board or another adapter material, the image quality is guaranteed. After the connection wire 200 is soldered, a wire protection layer 210 may be formed on an area corresponding to the wire connection member 130. The wire protection layer 210 and the light shield 600 may be designed to be an integral component and formed simultaneously in a single operation after the connection of the connection wire 200. Thus, the connection points between the connection wire 200 and the sensor 700, the image module 300 and the light source module 400 are also protected. Alternatively, the wire protection layer 210 and the light shield 600 may be fabricated separately, as shown in FIG. 2, wherein the wire protection layer 210 may be made of a UV-cured resin or a thermoset resin.

Refer to FIG. 12. In some embodiments, the image sensing device comprises two light source modules 400, the sensor 700, the optical protection layer 800, the light shield 600 and the wire protection layer 210, wherein the light shield 600 and the wire protection layer 210 are fabricated in a single process.

It is learned from the above discussion: the image sensing device has the following advantages:
1. The substrate 100 is used as the carrier to perform the panel-level fabrication process and package process and thus achieve the economic efficiency;
2. The carrier 500, which may have different heights, is used to control the relative height of the image module 300 and the light source module 400; the image module connection member 140 and the light source module connection member 150 are used to accurately control the distance between the image module 300 and the light source module 400, whereby are avoided the errors that are likely to occur in the conventional assembly operations;
3. The light shield 600 is filled into the area around the image module 300 and the light source module 400 to effectively prevent stray light from entering the image module 300 and protect the image module 300 and the light source module 400;
4. The connection wire 200 is not soldered onto the second surface 120 of the substrate 100 but is soldered onto the first surface 110 where the other elements are also disposed, whereby the size of the image sensing device and the size of the distal tip of the side-viewing endoscope are minimized;
5. The connection wire 200 is exempted from using an adapter board but is directly soldered onto the substrate 100, whereby to guarantee the completeness of signals, improve the signal-to-noise ratio, and enhance the image quality;
6. The sensor 700 is used to monitor the status inside the human body lest the human body be harmed; and
7. The image sensing device may be equipped with several light source modules 400, which can emit lights of different wavelengths and colors and can be switched to respectively provide different types of illuminations or simultaneously provide illuminations.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. An image sensing device for a side-viewing endoscope, the image sensing device being disposed in a distal tip of the endoscope and comprising:
a substrate, including a first surface and a second surface opposite to the first surface, and also including a wire connection member, an image module connection member, and a light source module connection member, which are all arranged on the first surface, wherein the wire connection member is electrically connected with the image module connection member; the wire connection member is also electrically connected with the light source module connection member;
an image module, electrically connected with and installed on the image module connection member, and capturing external images along a lateral-side direction of the distal tip, wherein the lateral-side direction of the distal tip is a direction parallel to the lateral side of the distal tip and substantially vertical to a first direction; and
at least one light source module, electrically connected with and installed on the light source module connection member, the least one light source module being configured to project light along lateral-side direction of the distal tip.

2. The image sensing device for a side-viewing endoscope according to claim 1, wherein the light source module connection member is disposed on the first surface and at positions including either side or both sides of the image module connection member.

3. The image sensing device for a side-viewing endoscope according to claim 1, further comprising a connection wire, wherein one end of the connection wire is electrically connected with the wire connection member; the wire connection member, the image module connection member, and the light source module connection member are arranged along a first direction; the first direction is parallel to a line from a first end of the first surface to a second end of the first surface, which is opposite to the first end.

4. The image sensing device for a side-viewing endoscope according to claim 3, wherein the light source module connection member is disposed on the first surface and at one side of the image module connection member, which is corresponding to the first end, along the first direction; the wire connection member is disposed on the first surface and at one side of the image module connection member, which is corresponding to the second end, along the first direction.

5. The image sensing device for a side-viewing endoscope according to claim 3, wherein the light source module connection member is disposed on the first surface and at one side of the image module connection member, which is corresponding to the second end, along the first direction; the wire connection member is disposed on the first surface and at one side of the image module connection member, which is corresponding to the first end, along the first direction.

6. The image sensing device for a side-viewing endoscope according to claim 3, wherein the light source module connection member is disposed on the first surface and at two sides of the image module connection member, which are corresponding to the first end and the second end, along the first direction; the wire connection member is disposed on the first surface and at a position between the light source module connection member and the first end; alternatively, the wire connection member is disposed on the first surface and at a position between the light source module connection member and the second end.

7. The image sensing device for a side-viewing endoscope according to claim 1, further comprising a carrier, which is disposed between the light source module and the light source module connection member.

8. The image sensing device for a side-viewing endoscope according to claim 7, further comprising an intermediate carrier, which is disposed between the carrier and the light source module.

9. The image sensing device for a side-viewing endoscope according to claim 1, further comprising a light shield, which are disposed between the light source module and the image module.

10. The image sensing device for a side-viewing endoscope according to claim 9, wherein the light shield is disposed around the light source module.

11. The image sensing device for a side-viewing endoscope according to claim 9, wherein the light shield is disposed around the image module.

12. The image sensing device for a side-viewing endoscope according to claim 9, wherein an optical protection layer is formed on a surface of the light source module.

13. The image sensing device for a side-viewing endoscope according to claim 1, wherein the light source module is a light-emitting diode or a combination of a fluorescent powder layer and a light-emitting element, which generates a white light source.

14. The image sensing device for a side-viewing endoscope according to claim 1, wherein the first surface further includes a sensor connection member; the sensor connection member is electrically connected with the wire connection member; a sensor is installed in the sensor connection member.

15. The image sensing device for a side-viewing endoscope according to claim 1, further comprising a connection wire, wherein one end of the connection wire is electrically connected with the wire connection member; a wire protection layer is disposed at a position of the connection wire, which is corresponding to the wire connection member.

16. The image sensing device for a side-viewing endoscope according to claim 1, wherein the light source module includes light-emitting elements for respectively generating lights with different wavelength; the wire connection member a control wire is connected with the light-emitting elements to receive control signals for controlling wavelengths.
